(19) United States
(12) Patent Application Publication
(10) Pub. No.: US 2024/0376410 A1
(43) Pub. Date: Nov. 14, 2024

(54) AMINE-BASED SURFACE CLEANER AND DISINFECTANT WITH FAST MICROBIOLOGICAL EFFICACY

(71) Applicant: ECOLAB USA INC., St. Paul, MN (US)

(72) Inventors: Michael Decker, Solingen (DE); Jasmin Weber, Monheim am Rhein (DE); Andreas Krause, Dusseldorf (DE)

(73) Assignee: ECOLAB USA INC., St. Paul, MN (US)

(21) Appl. No.: 18/659,725

(22) Filed: May 9, 2024

Related U.S. Application Data
(60) Provisional application No. 63/501,214, filed on May 10, 2023.

(51) Int. Cl. C11D 3/48; A01N 33/04; A01N 43/36; A01P 1/00; A61L 2/18; A61L 101/36; C11D 1/722; C11D 3/20; C11D 3/30; C11D 3/32; C11D 3/33

(52) U.S. Cl. CPC C11D 3/48; A01N 33/04; A01N 43/36; A01P 1/00; A61L 2/18; C11D 1/722; C11D 3/201; C11D 3/30; C11D 3/32; C11D 3/33; A61L 2101/36

(57) ABSTRACT

Antimicrobial compositions comprising at least an amine-based antimicrobial and an alkanolamine are disclosed. The disclosed compositions may be free of quaternary ammonium compounds. The disclosed compositions have antimicrobial properties and rapid efficacy against yeast and bacteria.

### CROSS-REFERENCE TO RELATED APPLICATION

This application claims the benefit of and priority to U.S. Provisional Application No. 63/501,214 filed on May 10, 2023, which is incorporated herein by reference.

### BACKGROUND

Disinfecting and sanitizing surfaces in sensitive areas such as hospitals, food and beverage facilities, food surfaces, food contact surfaces in restaurants and grocery stores, and cleanrooms is challenging. Antimicrobial compositions for use in such applications must achieve a high log reduction against a broad spectrum of microorganisms including gram positive and gram negative bacteria, bacterial spores, yeasts, fungi, and viruses. The desired log reduction must be achieved in a short amount of time. Depending on the application, the antimicrobial compositions must be able to meet additional criteria, such as not leaving behind a residue, being approved for food contact, or being approved for food surface contact. It is against this background that the present invention is made.

### SUMMARY

The present disclosure relates to a method of inactivating microorganisms by applying a use composition to a target surface. In some embodiments the composition is comprised of a biocidal amine, an alkanolamine, and a nonionic surfactant. In some embodiments the composition achieves a 3-log reduction against yeast within 5 minutes at 20° C. without mechanical action, and achieves a 4-log reduction against bacteria within 5 minutes as 20° C. without mechanical action. In some embodiments the use composition is free of quaternary ammonium compound. In some embodiments the use composition includes a complexing agent. In some embodiments the use composition has a pH within a range of about 10 to about 12.

The present disclosure also relates to a method of inactivating yeast by applying a composition to a target surface, where the method achieves a 3-log reduction against yeast within 5 minutes at 20° C. In some embodiments the composition is comprised of a biocidal amine, an alkanolamine, and a nonionic surfactant. In some embodiments, the method also achieves a 4-log reduction against bacteria within 5 minutes at 20° C. In some embodiments the use composition is free of quaternary ammonium compound. In some embodiments the use composition includes a complexing agent. In some embodiments the use composition has a pH within a range of about 10 to about 12.

The present disclosure also relates to a composition for inactivating yeast by applying a composition to a target surface, where the composition achieves a 3-log reduction against yeast within 5 minutes at 20° C. In some embodiments the composition is comprised of a biocidal amine, an alkanolamine, and a nonionic surfactant. In some embodiments, the composition also achieves a 4-log reduction against bacteria within 5 minutes at 20° C. In some embodiments the use composition is free of quaternary ammonium compound. In some embodiments the use composition includes a complexing agent. In some embodiments the use composition has a pH within a range of about 10 to about 12.

### BRIEF DESCRIPTION OF THE DRAWINGS

FIG. 1 depicts a chart detailing the results of a cleaning efficacy test.

### DETAILED DESCRIPTION

As used herein, weight percent (wt. %), percent by weight, % by weight, %, and the like are synonyms that refer to the concentration of a substance as the weight of that substance divided by the total weight of the composition and multiplied by 100.

As used herein, the term “about” modifying the quantity of an ingredient in the compositions of the invention or employed in the methods of the invention refers to variation in the numerical quantity that can occur, for example, through typical measuring and liquid handling procedures used for making use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about,” the claims include equivalents to the quantities. The term “about” typically allows for a variation within 5% of the stated percent value. For example, if “about 10%” is used, the term “about” typically allows for a variation of about 5% of the stated percent value. Thus, “about 10%” may cover a variation of +/−0.5%.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

As used herein, “clean” or “cleaning” refers to the removal or organic or inorganic soil. In some examples, “clean” or “cleaning” can refer to any process that aids in soil removal.

As used herein, the term “disinfect” or “disinfecting” refers to biological action that kills vegetative cells and reduces microbial populations, including most recognized pathogenic microorganisms.

As used herein, the term “microorganism” refers to any noncellular or unicellular (including colonial) organism. Microorganisms include all prokaryotes. Microorganisms include bacteria (including cyanobacteria), spores, lichens, fungi, protozoa, virions, viroids, viruses, phages, and some algae. As used herein, the term “microbe” is synonymous with microorganism.

In fields where cleanrooms are utilized, it is important to maintain a controlled environment, limiting or eliminating microbial growth such as bacteria, yeast, or fungi. Additionally, it is important for these environments to be free not only of microbial contamination, but also of visible stains or soil. As a result, the antimicrobial compounds must also have little to no foaming when applied and must leave minimal to no residue. Many commercially available antimicrobials rely on the use of quaternary ammonium disinfectants, which have high efficacy against gram-positive bacteria, but reduced efficacy against gram-negative bacteria and little to no effect on yeast and fungi.

One alternative method of eliminating microbial growth is the use of amine-based antimicrobial compounds. Previous amine based antimicrobial compositions are limited by factors including a limited microbiological efficacy profile, poor cleaning performance, incompatibility with target surfaces, and/or foaming or residue formation. Additionally, some existing antimicrobial compositions do not achieve the desired 3-log reduction within the target time of 2, 5, or 10 minutes of contact with the target surface.

Surprisingly, the disclosed combination of a biocidal amine with an alkanolamine has been found to be effective against yeasts in a short period of time. In addition to achieving the desired log reduction against yeasts in the desired time period, the disclosed formula is effective against a range of microorganisms including gram-negative and gram-positive bacteria and yeast, exhibits minimal foaming and residue formation, and is compatible with a range of surfaces including glass, polymers, PVC, pharmaceutical terrazzo, epoxy, and stainless steel. This compound is also free of quaternary ammonium compounds. The disclosed compounds are especially useful in cleanroom applications that have strict requirements, but are also useful for antimicrobial applications generally, including in healthcare and food and beverage applications.

### Active Ingredients

In some embodiments, the disclosed composition comprises an amine, an alkanolamine, a nonionic surfactant, and water. The disclosed composition may also include additional functional ingredients.

In some embodiments, the disclosed compositions consist essentially of an amine, an alkanolamine, a nonionic surfactant, and water. These compositions may also include additional functional ingredients. In this disclosure, the phrase “consisting essentially of” certain ingredients means a composition including those ingredients and lacking any ingredient that materially affects the basic and novel characteristics of the composition or method. The phrase “consisting essentially of” excludes from the claimed compositions and methods other materials that affect the antimicrobial efficacy of the composition unless such an ingredient is specifically listed after the phrase.

In some embodiments, the disclosed compositions consist of an amine, an alkanolamine, a nonionic surfactant, and water.

### Biocidal Amine

In some embodiments the amine present in the composition is a biocidal amine. Exemplary biocidal amines include N-(3-Aminopropyl)-N-dodecylpropane-1,3-diamine (e.g., CAS No.: 2372-82-9, commercially available as Lonzabac 12), glucoprotamine, or combinations thereof. In some embodiments, the biocidal amine is present in concentrations in the concentrate of about 2 wt. % to about 30 wt. %, about 2 wt. % to about 20 wt. %, about 2 wt. % to about 10 wt. %, about 5 wt. % to about 30 wt. %, about 10 wt. % to about 30 wt. %, or about 20 wt. % to about 30 wt. %. In some embodiments, the biocidal amine is present in concentrations in the use solution from about 0.01 wt. % to about 2 wt. %, from about 0.01 wt. % to about 5 wt. %, from about 0.01 wt. % to about 10 wt. %, from about 2 wt. % to about 10 wt. %, or from about 5 wt. % to about 10 wt. %.

### Alkanolamine

In some embodiments the composition also includes an alkanolamine for the purpose of boosting the performance of the antimicrobial against bacteria and especially yeast. Alkanolamines are compounds which contain hydroxyl- and amino-groups such as monoethanolamine, diethanolamine, triethanolamine, or dimethylethanolamine. In some embodiments, the alkanolamine is present in concentrations in the concentrate of about 2 wt. % to about 30 wt. %, about 2 wt. % to about 20 wt. %, about 2 wt. % to about 10 wt. %, about 5 wt. % to about 30 wt. %, about 10 wt. % to about 30 wt. %, or about 20 wt. % to about 30 wt. %. In some embodiments the alkanolamine is present in concentrations in the use solution from about 500 ppm to about 0.01 wt. %, from about 0.01 wt. % to about 2 wt. %, from about 0.01 wt. % to about 5 wt. %, from about 0.01 wt. % to about 10 wt. %, from about 2 wt. % to about 10 wt. %, or about 5 wt. % to about 10 wt. %

### Nonionic Surfactant

In some embodiments the disclosed composition also includes a nonionic surfactant. Exemplary nonionic surfactants include C₁₂ to C₁₄ fatty alcohol surfactants, C₁₂ to C₁₄ EO/PO copolymer surfactants, EO-endcapped surfactants, and alkylpolyglycoside surfactants. Fatty alcohol surfactants are a preferred surfactant for the present composition because it allows for suitable foam formation after the initial application but allows for the foam formation to decrease over time as is necessary for cleanroom environments. In some embodiments, the nonionic surfactant is present in concentrations in the concentrate of about 1 wt. % to about 30 wt. %, about 1 wt. % to about 20 wt. %, about 1 wt. % to about 10 wt. %, from about 5 wt. % to about 30 wt. %, from about 10 wt. % to about 30 wt. % or about 20 wt. % to about 30 wt. %. In some embodiments the nonionic surfactant is present in concentrations in the use solution from about 0.01 wt. % to about 2 wt. %, from about 0.01 wt. % to about 5 wt. %, from about 0.01 wt. % to about 10 wt. %, from about 2 wt. % to about 10 wt. %, or from about 5 wt. % to about 10 wt. %.

### Complexing Agent

In some embodiments the disclosed composition also includes a complexing agent. Exemplary complexing agents include an aminocarboxylate, EDTA, NTA, GLDA, MGDA, and mixtures thereof. A preferred complexing agent is methylglycinediacetic acid (MGDA). In some embodiments the complexing agent may be present in concentrations in the use solution from about 1 ppm to about 1000 ppm, from about 50 ppm to about 500 ppm, or from about 50 ppm to about 150 ppm.

### Water

The disclosed compositions are preferably liquids, thickened liquids, gels, or foams and include water. In some embodiments, the water is present in amounts from about 75 to about 99.9 wt. %, from about 85 to about 99.9 wt. %, from about 95 to about 99.9 wt. %, or from about 99.0 to about 99.9 wt. %. In some embodiments, the present composition is provided as a concentrated product intended for dilution prior to use. In some embodiments, the present composition is provided as a ready to use composition.

In some embodiments, the water is purified, such as water for injection (WFI). The water may also be deionized, distilled, sterile, or bacteriostatic. However, in some embodiments, the water used in the disclosed compositions is not purified or sterilized. In such examples, the water may be non-sterile, such as hard water or tap water that includes minerals such as calcium, sodium, magnesium, fluoride, copper, iron, chloride, and other minerals. With non-sterile sources of water, a chelating agent may be added to complex to the minerals and act as a scale inhibitor. In examples, the chelating agenting can be phosphorus based, aminocarboxylate based, or other form of chelating agent. Preferably, the chelating agent is a phosphorus based chelating agent, such as a phosphonate.

In some examples, the phosphorus-based chelating agent is a phosphonate. The phosphonate may be added to the concentrate in amounts from about 0.01 wt. % to about 2 wt. %, from about 0.01 wt. % to about 1 wt. %, from about 0.01 wt. % to about 0.75 wt. %, from about 0.01 wt. % to about 0.5 wt. %, from about 0.01 wt. % to about 0.1 wt. %, from about 0.1 wt. % to about 2 wt. %, from about 0.5 wt. % to about 2 wt. %, from about 0.75 wt. % to about 2 wt. %, or from about 1 wt. % to about 2 wt. %.

### Additional Functional Ingredients

In some embodiments, the antimicrobial compositions may optionally include additional functional ingredients. Exemplary additional functional ingredients include thickening or gelling agents, co-solvents, additional foaming agents, chelating agents, or mixtures thereof.

### Thickening or Gelling Agents

In some applications, it may be desirable for the current composition to have greater viscosity or to take the form of a thickened liquid or gel. Exemplary thickening agents include those which do not leave contaminating residue on the surface of application. Exemplary thickeners include natural gums such as xanthan gum. Also useful in the present invention are cellulosic polymers, such as carboxymethyl cellulose. Generally, the concentration of thickener use in the present invention will be dictated by the desired viscosity within the final composition.

In some embodiments, the composition has a viscosity that is similar to water or close to 0 centipoise. In some embodiments, the composition may be a concentrate composition or a ready to use composition with a viscosity from 0 to about 50 centipoise, from 0 to about 100 centipoise, from 0 to about 1000 centipoise, from about 50 to about 750 centipoise, or from about 100 to about 500 centipoise as determined by a Brookfield viscometer with spindle 61 at 100 rpm at 20° C.

### Co-Solvents

In some embodiments, the composition may include a co-solvent. Preferred co-solvents include glycols and C1-C4 alcohols such as butylene glycol, propanol, ethanol, methanol, or propylene glycol, among others.

### Additional Surfactants or Foaming Agents

While the current disclosure emphasizes the need for minimal foam in order to reduce residues, which are undesirable in cleanroom applications, in other applications additional foaming may be a desirable quality. The formation of foam can increase contact time on non-horizontal surfaces, allowing the active ingredients additional contact time with the target microbes to reach the desired log reduction.

pH

In some embodiments, the disclosed compositions have a pH in the range of about 7 to about 14, about 8 to about 13, about 9 to about 12, or about 10 to about 12.

### Absence of Quaternary Ammonium Compounds

In some embodiments, the disclosed composition contains no quaternary ammonium compounds. Quaternary ammonium compounds are a commonly used antimicrobial in cleaning solutions; however, some potential users prefer alternate antimicrobials such as the disclosed composition. Aversion to using quaternary ammonium compounds may be a result of the high cost of manufacturing and low efficacy against microbes other than gram-positive bacteria.

### Methods of Inactivating Microorganisms

The present disclosure relates to methods of inactivating microorganisms and in particular bacteria and yeast within a short period of time using the disclosed antimicrobial compositions. In some embodiments, the disclosed antimicrobial composition has a log reduction of greater than 4-log against bacteria within 2 minutes of contact, a log reduction of greater than 5-log against bacteria within 2 minutes of contact, a log reduction of 4-log to 6-log against bacteria within 2 minutes of contact, a log reduction of 4-log to 7-log against bacteria within 2 minutes of contact, a log reduction of 5.5-log to 6.5-log against bacteria within 2 minutes of contact, or a log reduction of 6-log to 6.5-log against bacteria within 2 minutes of contact at room temperature and without mechanical action. The exemplary test organisms includes S. aureus, E. hirae, E. coli, and P. aeruginosa under EN 13697.

In some embodiments, the disclosed antimicrobial composition has a log reduction of 3-log or greater against yeast within 2 minutes of contact, a log reduction of 2-log to 5-log against yeast within 2 minutes of contact, a log reduction of 3-log to 4-log against yeast within 2 minutes of contact, or a log reduction of 3.5-log to 4-log against yeast within 2 minutes of contact at room temperature on a surface without mechanical action under EN 13697.

In some embodiments, the disclosed antimicrobial composition has a log reduction greater than 3-log against fungi within 5 minutes of contact, a log reduction greater than 3.5-log against fungi within 5 minutes of contact, a log reduction greater than 4-log against fungi within 5 minutes of contact at room temperature, a log reduction of 3-log to 5-log against fungi within 5 minutes of contact at room temperature on a surface without mechanical action under EN 13697.

### Microbial Strains

A variety of microbes were used in testing to confirm the efficacy of the disclosed composition. Among those microbes tested were gram-positive and gram-negative bacterial strains. Gram-positive bacterial strains used included Enterococcus hirae and Staphylococcus aureus. Gram negative strains used included Escherichia coli and Pseudomonas aeruginosa. Yeast strain Candida albicans was also tested, as was fungal strain Aspergillus brasiliensis. These strains are a sampling of potential species against which the current composition may be utilized, and are by no means comprehensive of the current composition's antimicrobial efficacy. The test used was EN 13697.

### Applications

In some embodiments, the disclosed compositions are applied to hard surfaces located in research areas, manufacturing facilities, laboratories, cleanrooms, hospitals, resorts, hotels and motels, commercial real estate, schools, and food or drink manufacturers, among others. Amine based antimicrobials are ideal for hard surfaces which are susceptible to microbial contamination or growth, but which do not have high soil levels, as amines tend to break down and lose functionality when exposed to soils. As such, the present composition is beneficial for use in cleanrooms, pharmaceutical manufacturing facilities, laboratories, schools, resorts, hotels, and motels, commercial real estate, hospitals, and other areas where soil is minimal. Exemplary hard surfaces include counters, sinks, walls, drains, floors, ceilings, tables, carts, benches, transfer hatches, pass-through hatches, storage areas, cutting boards, blades, lab equipment, lab instruments, tanks, pipes, evaporators, membranes, heat exchangers, spray heads, nozzles, controllers, tools, handles, remotes, toilet handle, a toilet seat, a bedpan flushing device, a faucet handle, a doorknob or door handle, a push plate, a grab plate, a toilet area grab bar, a telephone receiver, a call button, a table, curtains, restricted access barrier systems (RABS), clean room equipment garments, personal protection equipment, laboratory hoods, safety cabinets, laminar flow cabinets, clean room isolators, a chair seat, a chair arm, a bedrail, a drape, a room light switch, a computer mouse, a computer keyboard, a keyboard wrist rest, a soap dispenser, among others.

### EXAMPLES

### Example 1

Example 1 tested the efficacy of various formulas against a variety of microbes including bacteria and yeast. During the test, various undiluted formulas were put in contact with a variety of bacterial strains and the yeast strain Candida albicans at room temperature under clean conditions according to test procedure EN 13697 for a period of 5 or 10 minutes. In order to be considered as having bactericidal activity, a formula was required to demonstrate at least a 4-log reduction after 5 minutes of exposure with a maximum of 60 minutes of exposure. In order to be considered as having yeasticidal activity formula was required to demonstrate at least a 3-log reduction after five minutes of exposure with a maximum of 60 minutes of exposure. The Experimental Formula 1, listed in Table 1, was tested and compared against Klercide Low Residue Quat, a commercially available product from Ecolab. The results are shown in Table 1.

### Experimental Formula 1

| | | | |
|---|---|---|---|
| Lonzabac 12 | biocidal amine | 0.16 | wt. % |
| Dehypon LS 54 | fatty alcohol (C12-C14) | 0.1 | wt. % |
| | EO/PO nonionic | | |
| | surfactant | | |
| | monoethanolamine | 0.16 | wt. % |
| | n-propanol | 0.075 | wt. % |
| Trilon M liquid | methylglycinediacetic | 0.01 | wt. % |
| | acid chelating agent | | |

TABLE 1

| Product | Bacteria | Bacteria | Yeast | Yeast |
|---|---|---|---|---|
| Contact Time | 5 Minutes | 10 minutes | 5 Minutes | 10 minutes |
| Klercide Low | 2.78 | 2.22 | 1.65 | 0.78 |
| Residue Quat | | | | |
| (Ecolab Inc.) | | | | |
| Experimental | 4.26 | n/a | 5.36 | n/a |
| Formula 1 | | | | |

The data shown in Table 1 demonstrates the Experimental Formula 1 was able to obtain the desired 4-log reduction against bacteria after 5 minutes of exposure and the desired 3-log reduction against yeast after five minutes of contact.

### Example 2

Example 2 tested the foaming performance of the Experimental Formula 1 over time. For this test, 100 ml of Experimental Formula 1 was placed in a 250 ml cylinder and shaken by hand 20 times in even, rhythmic movements. The height of the foam generation was measured on the cylinder immediately after shaking the cylinder, 1 minute after shaking the cylinder, and five minutes after shaking the cylinder. The results of this experiment are shown in Table 2.

TABLE 2

| Time (Min) | Foam Formation (mm) |
|---|---|
| 0 | 90 |
| 1 | 80 |
| 5 | 35 |

The results shown in Table 2 demonstrate some foam formation immediately upon application. These results also demonstrate a rapid decrease in foam as time passes. These results are desirable for cleanroom applications as some foam formation helps support cleaning, but excess foam formation can result in residue or stains.

### Example 3

Example 3 tested the residue profile of various formulas on a stainless steel surface as a result of spraying and wiping. When sprayed on stainless steel and left to evaporate, the experimental formula left a slight residue.

| | Residue on | |
|---|---|---|
| Formula | Evaporation (ppm) | |
| Experimental Formula 1 | 2700 | ppm |
| Klercide Low Residue Quat | 400 | ppm |
| (Ecolab Inc.) | | |
| Klercide Sporicidal Active Chlorine | 19,000 | ppm |
| (Ecolab Inc.) | | |

When sprayed on glass, or when wiped on either glass or stainless steel and left to evaporate, no residue was left.

### Example 4

Example 4 tested the cleaning profile of various formulas. Along with Experimental Formula 1, two commercial formulas were tested, Klercide Neutral Detergent and Anios Clean Surf, both commercially available from Ecolab. These formulas were tested against a tap water control.

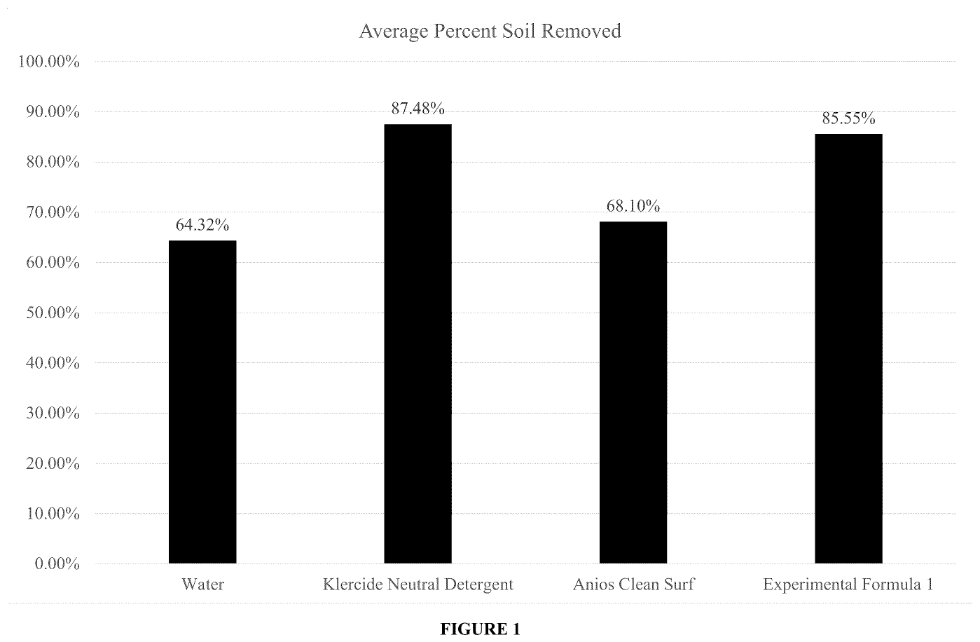

This test was completed using a Gardner abrasion testing (Gardner Apparatus Model 494; DIN-ASTM-515; Erichsen GmbH & Co. KG). For this test, white PVC coupons were coated with 2 grams of soil (IPP 83/21 available from WFK, Krefeld, Germany). 12.5 ml of the test detergent or the tap water control were applied to the test strips and another 12.5 ml of the test detergent or control was added to the sponge on the device for a total of 25 ml per test cycle. The test strips were then abraded for 100 back and forth cycles across the surface of the coated test strip using a polyester flat sponge. Following the completion of the test, the test strips were removed from the machine, rinsed, dried, and evaluated determined how clean they were by measuring the whiteness of the test strip. The whiteness of the coupons was measured with a Minolta Chroma Meter CR-200 and the percentage of the soil removed was recorded. If the coupon was completely clean (all of the soil removed), the test measured 100%. Seven replicates were completed for each cleaning solution. The results of this experiment are shown as percent clean in Table 3 and FIG. 1.

TABLE 3

| | | Klercide | | |
|---|---|---|---|---|
| | | Neutral | Anios Clean | Experimental |
| | Water | Detergent | Surf | Formula 1 |
| Test Strip 1 | 66.73% | 79.77% | 69.55% | 86.74% |
| | 67.18% | 80.60% | 67.72% | 86.28% |
| | 65.41% | 81.78% | 68.61% | 86.69% |
| | 64.64% | 80.79% | 68.13% | 86.15% |
| | 64.72% | 82.54% | 67.42% | 86.00% |
| | 61.04% | 81.99% | 67.99% | 86.15% |
| | 66.94% | 81.79% | 68.00% | 87.11% |
| Test Strip 2 | 60.24% | 90.23% | 67.43% | 86.01% |
| | 60.17% | 90.49% | 67.86% | 86.12% |
| | 62.72% | 90.63% | 66.00% | 86.59% |
| | 66.27% | 90.41% | 67.29% | 86.32% |
| | 63.38% | 90.00% | 68.00% | 87.00% |
| | 63.64% | 90.21% | 68.22% | 86.77% |
| | 60.88% | 89.56% | 67.89% | 86.71% |
| Test Strip 3 | 66.73% | 91.83% | 69.66% | 84.33% |
| | 67.18% | 91.24% | 68.66% | 83.00% |
| | 65.41% | 90.43% | 69.00% | 83.16% |
| | 64.64% | 90.49% | 68.19% | 83.75% |
| | 64.72% | 90.00% | 68.11% | 84.00% |
| | 61.04% | 91.22% | 69.01% | 83.77% |
| | 66.94% | 91.11% | 67.88% | 84.00% |
| Average | 64.32% | 87.48% | 68.10% | 85.55% |

The results shown in Table 3 and FIG. 1 show the experimental formula has an average cleaning efficacy of 85.55%. The highest average efficacy was seen with the commercial formula Klercide Neutral Detergent, which had an average efficacy of 87.48%. Of the formulas tested, Anios Clean Surf had the lowest average efficacy at 68.10%, and the control had an average cleaning efficacy of 64.32%. This data demonstrates that the disclosed composition has higher or comparable cleaning efficacy to other commercially available cleaning products.

### Example 5

Examples 1-4 tested the performance of Experimental Formula 1 under low soiling conditions. Example 5 compared the performance of Experimental Formula 1 under low soiling conditions and high soiling conditions.

Table 4 shows the results of testing Experimental Formula 1 under low soiling and high soiling conditions. Eighteen total tests were performed. Experimental Formula 1 was evaluated under low soiling conditions as a bactericide, a yeasticide, and a virucide against enveloped viruses. Each of these three functions of Experimental Formula 1 was tested in a suspension test, a surface test without mechanical action, and a surface test with mechanical action. For each of these variables, the contact time in minutes was measured. The time periods in Table 4 represent the number of minutes required for Experimental Formula 1 to satisfactorily perform as a bactericide, yeasticide, and virucide in accordance with the requirements of each of the three tests. The same was performed under high soiling conditions.

TABLE 4

| | | Low soiling | High soiling |
|---|---|---|---|
| | | conditions | conditions |
| Bactericide | Suspension test | 1 | 1 |
| (contact time | Surface test w/out | 2 | 2 |
| in minutes) | mechanical action | | |
| | Surface test w/ | 1 | 1 |
| | mechanical action | | |
| Yeasticide | Suspension test | 1 | 1 |
| (contact time | Surface test w/out | 2 | 2 |
| in minutes) | mechanical action | | |
| | Surface test w/ | 1 | 1 |
| | mechanical action | | |
| Virucide against | Suspension test | 1 | 1 |
| enveloped viruses | Surface test w/out | 5 | 10 |
| (contact time | mechanical action | | |
| in minutes) | Surface test w/ | 1 | 1 |
| | mechanical action | | |

The results in Table 4 show that Experimental Formula 1 provides substantially the same cleaning in both low soiling and high soiling conditions. For example, Experimental Formula 1 suitably acts as a bactericide within the same time period under both low soiling and high soiling conditions. This is advantageous because Experimental Formula 1 may be used as a bactericide, yeasticide, and virucide in both low and high soiling applications, which increases the applicability of the formula.

The above specification, examples, and data provide a complete description of the manufacture and use of the composition of the invention. Since many embodiments of the invention can be made without departing from the spirit and scope of the invention, the invention resides in the claims hereinafter appended.

What is claimed is:

1. A method of inactivating microorganisms comprising applying a use composition to a surface, the use composition comprising:
a. from about 0.01 to about 2 wt. % of a biocidal amine;
b. from about 0.01 to about 2 wt. % of an alkanolamine; and
c. from about 0.01 to about 2 wt. % of a nonionic surfactant.

2. The method of claim 1, wherein the use composition achieves a 3-log reduction against Candida albicans within 5 minutes at 20° C. without mechanical action.

3. The method of claim 1, wherein the use composition achieves a 4-log reduction against Enterococcus hirae within 5 minutes at 20° C. without mechanical agitation.

4. The method of claim 1, wherein the use composition is free of a quaternary ammonium compound.

5. The method of claim 1, wherein the biocidal amine is selected from the group consisting of N-(3-Aminopropyl)-N-dodecylpropane-1,3-diamine or glucoprotamine.

6. The method of claim 1, wherein the alkanolamine is selected from the group consisting of monoethanolamine, diethanolamine, triethanolamine, dimethylethanolamine, and combinations thereof.

7. The method of claim 1, wherein the nonionic surfactant is selected from the group consisting of ethylene oxide/propylene oxide copolymers, ethylene oxide endcapped surfactants, alkylpolyglycosides, and mixtures thereof.

8. The method of claim 1, wherein the nonionic surfactant is a C₁₂-C₁₄ EO/PO surfactant.

9. The method of claim 1, the composition further comprising a complexing agent selected from the group consisting of an aminocarboxylate, EDTA, NTA, GLDA, MGDA, and mixtures thereof.

10. The method of claim 1, wherein the use composition has a pH of about 10 to about 12.

11. A composition for inactivating yeast comprising:
a. from about 0.01 to about 2 wt. % of a biocidal amine;
b. from about 0.01 to about 2 wt. % of an alkanolamine; and
c. from about 0.01 to about 2 wt. % of a nonionic surfactant;
wherein the composition achieves a 3-log reduction against Candida albicans within 5 minutes at 20° C.

12. The composition of claim 11, wherein the composition achieves a 4-log reduction against Enterococcus hirea within 5 minutes at 20° C.

13. The composition of claim 11, wherein the use composition is free of a quaternary ammonium compound.

14. The composition of claim 11, wherein the biocidal amine is selected from the group consisting of N-(3-Aminopropyl)-N-dodecylpropane-1,3-diamine, glucoprotamine, and combinations thereof.

15. The composition of claim 11, wherein the alkanolamine is selected from the group consisting of monoethanolamine, diethanolamine, triethanolamine, dimethylethanolamine, and combinations thereof.

16. The composition of claim 11, wherein the nonionic surfactant is selected from the group consisting of ethylene oxide/propylene oxide copolymers, ethylene oxide endcapped surfactants, alkylpolyglycosides, and mixtures thereof.

17. The composition of claim 11, wherein the nonionic surfactant is a C₁₂-C₁₄ EO/PO surfactant.

18. The composition of claim 11, the composition further comprising a complexing agent selected from the group consisting of an aminocarboxylate, EDTA, NTA, GLDA, MGDA, and mixtures thereof.

19. The composition of claim 11, further comprising a C1 to C4 alcohol.

20. The composition of claim 11, wherein the composition has a pH of about 10 to about 12.
